(19) United States
(12) Patent Application Publication
(10) Pub. No.: US 2024/0415581 A1
(43) Pub. Date: Dec. 19, 2024

(54) METHOD FOR PROVIDING A CURVATURE PARAMETER, MEDICAL OBJECT AND SYSTEM

(71) Applicant: Siemens Healthineers AG, Forchheim (DE)

(72) Inventors: Alois Regensburger, Poxdorf (DE); Sebastian Schäfer, Bayern (DE)

(21) Appl. No.: 18/747,377

(22) Filed: Jun. 18, 2024

(30) Foreign Application Priority Data
Jun. 19, 2023 (DE) 10 2023 205 716.6

(51) Int. Cl. A61B 34/20; A61B 34/10; A61B 90/00

(52) U.S. Cl. CPC A61B 34/20; A61B 2034/107; A61B 2034/2065; A61B 2090/3764; A61B 2090/3983

(57) ABSTRACT

A method for provision of a curvature parameter includes capturing of medical imaging data by a medical imaging device, having an image of a medical object that is arranged at least partly in an examination object. At least one distal section of the medical object is able to be curved parallel to a curvature plane. The method includes identifying a positioning of the curvature plane with the aid of the image data, and providing a curvature parameter having an indication for positioning of the curvature plane.

This application claims the benefit of German Patent Application No. DE 10 2023 205 716.6, filed on Jun. 19, 2023, which is hereby incorporated by reference in its entirety.

### BACKGROUND

The present embodiments relate to providing a curvature parameter.

In interventional procedures (e.g., a bone ablation), it may be advantageous, for protecting sensitive regions of an anatomy of an examination object, to realize curved needle paths for access to a target object. Actively articulated medical objects (e.g., an articulated needle) may be articulated, for example, by a proximally arranged operating element. The operating element may have a display of the curvature setting of the medical object at that moment. Disadvantageously, the curvature setting shown may deviate from an actual curvature of a distal section of the medical object that is arranged in the examination object. An image-guided forwards movement of the articulated medical object (e.g., an articulated needle) along a planned curve is, however, challenging and often not possible with a desired precision. For example, with an as yet unarticulated arrangement of the medical object in the examination object and/or 2D imaging (e.g., a fluoroscopy), for guidance, a precise forwards movement may be rendered more difficult, since there is no information about direction of curvature and/or depth.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, better navigation support in the positioning of articulatable medical objects is provided.

Independent of the grammatical term usage, individuals with male, female, or other gender identities are included within the term.

In a first aspect, the present embodiments relate to a method for provision of a curvature parameter. In a first act a), medical image data, having an image of a medical object, with the medical object being at least partly arranged in an examination object, is captured by a medical imaging device. In this case, at least one distal section of the medical object is able to be curved parallel to a curvature plane. In a further act b), a positioning of the curvature plane is identified with the aid of the image data. In a further act c), a curvature parameter, having an indication for positioning of the curvature plane, is provided.

The examination object may, for example, be a female human and/or animal patient and/or a male human and/or animal patient and/or an examination phantom.

The medical object may be a surgical instrument (e.g., a needle, such as a puncture needle, and/or a drill, and/or a diagnostic instrument, such as an endoscope or a laparoscope, and/or a catheter). In one embodiment, the medical object may be configured at least partly (e.g., completely) elongated (e.g., in the shape of a rod and/or needle). At least one distal section of the medical object may further be able to be curved parallel to a curvature plane (e.g., using a cable). The distal section may be a spatial section facing towards the examination object (e.g., an end section) of the medical object (e.g., a tip). The medical object may further have a proximal section that is arranged facing away from the examination object (e.g., at a distance from the examination object).

The curvature plane may refer to a plane (e.g., a two-dimensional (2D) plane). The distal section of the medical object is able to be curved parallel to the curvature plane, especially within the curvature plane. For example, the medical object may have a defined curvature axis, about which the distal section is able to be curved.

In one embodiment, the distal section of the medical object may be able to be curved by manipulation of an operating element (e.g., at a proximal section of the medical object). For this, the medical object may have an adjustment element (e.g., a mechanical and/or electromagnetic and/or pneumatic adjustment element) that is configured to curve the distal section parallel to the curvature plane. For example, the medical object may have a cable (e.g., a Bowden cable) as its adjustment element. The adjustment element may be able to be manipulated by the operating element by a medical operator and/or a robotic movement apparatus.

In one embodiment, the curvature of the distal section may be controlled (e.g., by the adjustment element). The example, the curvature of the distal section may be able to be restricted and/or be able to be limited such that, during manipulation by the operating element, the distal section is able to be curved (e.g., only) parallel to the curvature plane.

In one embodiment, the distal section of the medical object may have been arranged before the beginning of the method in the examination object (e.g., in an examination region within the examination object). The examination region may, for example, include an anatomical structure of the examination object (e.g., a tissue and/or an organ, such as a hollow organ).

The capturing of the image data may include a receipt and/or acceptance of the image data. The receipt of the image data may, for example, include a capture and/or reading out of a computer-readable data memory and/or a receipt from a data memory unit (e.g., a database). The image data may further be provided by a provision unit of a medical imaging device. As an alternative or in addition, the image data may be recorded by the medical imaging device.

The medical imaging device for recording the image data may be a medical X-ray device (e.g., a medical C-arm X-ray device and/or a cone-beam computed tomography device (CT, CBCT)) and/or a computed tomography (CT) system and/or a magnetic resonance tomography (MRT) system and/or a positron emission tomography (PET) system and/or an ultrasound device.

The image data may have an image of the medical object that is arranged at least in part in the examination object. For example, the image data may depict at least the distal section of the medical object in the examination object. In one embodiment, the image data may be a two-dimensional (2D) and/or three-dimensional (3D) spatially resolved image of the medical object in the examination object. The 2D spatially resolved image may depict the medical object in the examination object as a 2D projection. For example, the image of the medical object may have a number of image points (e.g., pixels and/or voxels) with image values (e.g., attenuation values and/or intensity values) that depict the medical object (e.g., represent the medical object). The image data may further be time-resolved. For example, the dataset may have a time-resolved image and/or a time-resolved model of the number of anatomical and/or medical objects of the examination object.

In act b), the positioning (e.g., a spatial alignment and/or orientation and/or location and/or position and/or pose) of the curvature plane is identified with the aid of the image data.

In accordance with a first variant, the image data may depict the distal section in an unmanipulated (e.g., pre-curved or essentially straight) state. In this case, the positioning of the curvature plane may be identified with the aid of geometrical features of the medical object (e.g., a contour and/or a curvature marker structure and/or a contrast progression that is depicted in the image data). For example, the positioning of the curvature plane may be identified with the aid of asymmetrical geometrical features of the medical object. The geometrical features in this case may be characteristic for the positioning of the curvature plane (e.g., at that moment). The medical object (e.g., the distal section) may have a pre-curvature with defined relative alignment (e.g., defined angle) to the curvature plane of the distal section. The positioning of the curvature plane may be identified with the aid of an image (e.g., a 2D image) of the pre-curvature in the image data. As an alternative or in addition, the medical object may have an asymmetrical operating element for manipulation of the curvature of the distal section and/or an asymmetrical adjustment element for adjusting the curvature of the distal section. The positioning of the curvature plane is identified with the aid of geometrical features of the operating element and/or of the adjustment element, which are depicted in the image data.

In a second variant, the image data may depict the distal section in a curved state. In this case, the positioning of the curvature plane may be identified with the aid of a positioning of the curved distal section depicted in the image data and/or with the aid of geometrical features of the medical object (e.g., the contour and/or the curvature marker structure and/or the contrast progression) that are depicted in the image data. The geometrical features may be characteristic in this case for the positioning of the curvature plane (e.g., at that moment). On curvature, the distal section may have a defined geometrical shape (e.g., a circle segment shape and/or ellipse segment shape) parallel to the curvature plane. The positioning of the curvature plane may be identified with the aid of an image (e.g., a 2D image) of the curved distal section and knowledge about its defined geometrical shape.

In one embodiment, the curvature parameter may have information (e.g., quantitative information) about the positioning of the curvature plane at that moment (e.g., a graphical representation and/or a relative or absolute positioning specification; coordinates and/or a normal vector).

The provision of the curvature parameter may include storage on a computer-readable memory medium (e.g., a non-transitory computer-readable storage medium) and/or a display on a visual display unit and/or a transmission to a provision unit. For example, a graphical display of the curvature parameter may be shown by the visual display unit.

The method of the present embodiments, through provision of the curvature parameter, may make improved navigation support possible in the positioning of articulatable medical objects.

In a further form of embodiment of the method, a check may be made in act b) as to whether an imaging direction of the imaging device is aligned essentially at right angles to the curvature plane. If not, a relative positioning between the medical object and the imaging device may be adjusted, and the acts a) and b) may be carried out repeatedly.

In one embodiment, the positioning of the curvature plane with regard to a coordinate system of the imaging device is identified (e.g., by a registration). This enables a relative positioning (e.g., a relative position and/or relative alignment and/or relative pose) between the imaging direction of the imaging device and the curvature plane to be identified. In one embodiment, if it cannot be, especially on alignment of the imaging direction parallel or at an angle deviating from 90 degrees to the curvature plane, the relative positioning is adjusted and the acts a) and b) are carried out repeatedly. The adjustment of the relative positioning may include a repositioning of the imaging device with regard to the medical object and of the examination object and/or a repositioning of the medical object. In one embodiment, the relative positioning may be adjusted such that the imaging direction and the curvature plane are essentially at a right angle (e.g., at 90 degrees) to one another. If the imaging direction and the curvature plane are (e.g., with an essentially right angle alignment of the imaging direction of the imaging device to the curvature plane), the method may be continued with act c).

The form of embodiment may provide the identifiability of the positioning of the curvature plane with the aid of the image data and the provision of the curvature parameter through the adjustment of the relative positioning.

In a further form of embodiment of the method, act b) may include an identification of a positioning of a curvature marker structure on the medical object that is depicted in the image data.

The curvature marker structure may include one or more curvature markers visible to imaging (e.g., opaque to X-rays). The at least one curvature marker may have a predefined geometrical characteristic (e.g., a shape and/or contour) and/or structural characteristic (e.g., a material distribution and/or material density and/or material thickness). With the aid of an image of the at least one curvature marker and of its predefined geometrical characteristic and/or structural characteristic, the positioning of the curvature plane may be identified. As an alternative or in addition, the number of curvature markers may have a predefined relative positioning (e.g., a defined arrangement relative to one another). The positioning of the curvature plane may be identified with the aid of an image of the number of curvature markers and their predefined relative positioning.

The curvature marker structure may be arranged on the medical object (e.g., the distal section; attached to the medical object) and/or be integrated, at least partly (e.g., completely), into the medical object (e.g., the distal section; as a recess and/or raised area on a surface of the medical object).

The identification of the curvature marker structure (e.g., of the at least one curvature marker) may include an identification (e.g., segmentation) of image points (e.g., pixels and/or voxels) of the image data that depict the curvature marker structure (e.g., with the aid of a comparison of image values of the image points with a predetermined threshold value and/or with the aid of a shape of the curvature marker structure depicted).

The form of embodiment may make an improved (e.g., more precise) identification of the positioning of the curvature plane possible.

In a further form of embodiment of the method, a curvature of the distal section of the medical object at that moment may be identified with the aid of the positioning of the curvature marker structure. In this case, the curvature parameter having information about the curvature at that moment may be provided.

The identification of the curvature of the distal section at that moment may include an identification of a curvature direction and/or a curvature angle and/or a curvature path and/or a curvature geometry of the distal section of the medical object. For example, the identification of the curvature at that moment may be undertaken quantitatively.

In one embodiment, the curvature marker structure may be configured to represent the curvature of the distal section of the medical object at that moment (e.g., to display the curvature). For example, the curvature marker structure (e.g., the at least one curvature marker) due to its arrangement on the distal section may represent a course of the curvature (e.g., the curvature path). For this, a number of curvature markers may be arranged in a longitudinal extent direction of the distal section. Due to a relative positioning of the number of curvature markers in relation to one another depicted in the image data, the curvature of the distal section at that moment depicted may be identified. As an alternative or in addition, at least one curvature marker may be arranged so as to be able to be curved similarly on the distal section (e.g., attached or integrated). With the aid of the image of the at least one curved curvature marker in the image data, the curvature of the distal section at that moment may be identified. The curvature of the distal section at that moment may, for example, be identified with the aid of geometrical features of the curvature marker structure that are depicted in the image data (e.g., a contour).

In one embodiment, the curvature parameter may include information about the curvature of the distal section at that moment (e.g., a curvature direction and/or a curvature angle and/or a curvature path and/or a curvature geometry). The curvature parameter may, for example, have a graphical representation and/or a relative or absolute numerical curvature value.

Using the proposed form of embodiment, the curvature parameter having the information about the curvature of the distal section at that moment may be provided.

In a further form of embodiment of the method, the curvature marker structure may have a display element that is depicted in the image data and quantifies the curvature of the distal section at that moment. In this case, the provision of the curvature parameter may include a display of a graphical representation of the image data and of the curvature parameter by a visual display unit.

In one embodiment, the medical object may have an adjustment element that is configured for adjusting the curvature of the distal section. The adjustment element may further have an indicator structure visible to imaging (e.g., opaque to X-rays) as its display element. In this case, a relative positioning between the indicator structure and the curvature marker structure may change during an adjustment of the curvature of the distal section. The relative positioning between the indicator structure and the curvature marker structure may quantify the curvature of the distal section at that moment.

For example, the indicator structure may have a scale (e.g., labeling and/or line scale and/or a point scale), and the curvature marker structure may have a pointer (e.g., an arrow and/or a line and/or a point), or vice versa. In this case, the positioning of the pointer at that moment with regard to the scale may quantify the curvature at that moment. For example, the scale or the pointer may be arranged on the adjustment element (e.g., attached to the adjustment element), or may be integrated at least in part (e.g., completely) into the adjustment element. For example, the relative positioning between the indicator structure and the curvature marker structure may optically quantify the curvature of the distal section at that moment.

Using the form of embodiment, the curvature parameter may be provided having the quantitative information about the curvature of the distal section at that moment.

In a further form of embodiment of the method, the graphical representation of the curvature parameter may include a virtual continuation of the distal section in accordance with its curvature at that moment.

in one embodiment, the curvature parameter may include information about the curvature of the distal section at that moment (e.g., a curvature direction and/or a curvature angle and/or a curvature path and/or a curvature geometry). In this case, the virtual continuation of the distal section may be determined based on the curvature direction and/or the curvature angle and/or the curvature path and/or the curvature geometry. The virtual continuation may have a virtual representation (e.g., a line and/or a graphical model) of the distal section that continues a course of the medical object in this longitudinal extent direction virtually in accordance with the curvature at that moment. The virtual continuation of the distal section may be provided, for example, by an extrapolation of a curvature path (e.g., of a needle trajectory) based on a tangent of the distal section of the medical object in accordance with the curvature at that moment. In one embodiment, the graphical representation of the curvature parameter may have the virtual continuation of the distal section in accordance with its curvature at that moment also in a depth dimension with regard to a 2D representation plane of the visual display unit.

By the graphical representation of the curvature parameter, having the virtual continuation of the distal section in accordance with its curvature at that moment, a medical operator may be provided with help in the further navigation of the medical object.

In a further form of embodiment of the method, a material parameter for the medical object may be captured. In this case, the virtual continuation may be provided based on the material parameter and the curvature at that moment.

The material parameter may have (e.g. section-by-section or globally) information about a deformability (e.g., directed deformability; a deformability and/or stiffness and/or extensibility and/or torsional strength and/or bendability and/or elasticity) of the medical object. For example, the material parameter may include a tensor. The virtual continuation may be provided based on the material parameter and the curvature at that moment. In this case, a curvature angle and/or a curvature path of the virtual continuation may be extrapolated with the aid of the material parameter and the curvature at that moment.

The proposed form of embodiment may make a more precise provision of the virtual continuation possible.

In a further form of embodiment of the method, pre-operative planning data of the examination object may be received. In this case, the pre-operative planning data may be registered with the image data. Further, the provision of the curvature parameter may include a display of a graphical representation of the registered planning data and of the curvature parameter.

The planning data may include planning information about positioning of the medical object (e.g., path planning and/or target object and/or a required positioning and/or a required curvature for the medical object). The planning data may further have a pre-operative planning image of the examination object.

The registration of the planning data with the image data may include an application of a transformation specification to the planning data and/or the image data. The transformation specification may include an instruction for translation and/or rotation and/or scaling and/or deformation of the planning data and/or image data. In one embodiment, the registration may minimize a deviation between common anatomical and/or geometrical features that are contained (e.g., depicted and/or modeled) in the planning data and also the image data. The registration of the image data with the planning data may be based on the common anatomical and/or geometrical features and/or on an operating parameter (e.g., a recording parameter and/or reconstruction parameter) for provision of the planning data and for capture of the image data in each case.

In one embodiment, the provision of the curvature parameter may include a display of a graphical representation of the pre-operative planning data and of the curvature parameter. In this case, the registered planning data may be displayed, for example, in at least partial (e.g., complete) overlaying and/or mixing with the image data in the graphical representation. This enables an improved navigation support to be made possible.

The planning data may have a specification for a required curvature of the distal section. In this case, the curvature parameter may have information about a match or deviation between the curvature of the distal section at that moment and a required curvature. For example, the provision of the curvature parameter may include the provision of a graphical representation of a respective virtual continuation of the distal section in accordance with the curvature at that moment and in accordance with the required curvature. This enables a visual comparison between the curvature at that moment and the required curvature to be made possible.

In a further form of embodiment of the method, the planning data may include a pre-operative planning image of the examination object. In this case, the graphical representation of the planning data may include a graphical representation of the registered planning image. Further, the provision of the curvature parameter may also include a display of a graphical representation of the image data.

The pre-operative planning image may have a pre-operative image and/or a model (e.g., a volume network model and/or a skeletonized model) of the examination object. The pre-operative image may have been recorded pre-operatively by a medical imaging device. The model may have been provided, for example, with the aid of the pre-operative image of the examination object and/or by adjustment of a statistical and/or standardized patient model with the aid of patient parameters of the examination object. In one embodiment, the pre-operative planning image may depict and/or model the examination object spatially resolved in 2D or 3D. Further, the pre-operative planning image may be time resolved.

In one embodiment, the provision of the curvature parameter may include a display of a graphical representation of the image data and of the registered planning image. In this case, the registered planning image may be displayed in at least partial (e.g., complete) overlaying and/or mixing with the image data in the graphical representation.

This enables an improved navigation support to be made possible with regard to the pre-operative depiction of the examination object.

In a further form of embodiment of the method, act c) may include a provision of a workflow note that has an instruction for repositioning the curvature plane by repositioning the medical object and/or an instruction for adjusting the curvature of the distal section.

The workflow note may be provided by an output unit (e.g., optical and/or acoustic and/or haptic output unit; a visual display unit and/or a loudspeaker). The workflow note may include an optical and/or acoustic and/or haptic signal. For example, the workflow note may have a text form and/or spoken form and/or symbol form and/or graphics.

In accordance with a first variant, the workflow note may have an instruction (e.g., a movement specification and/or control specification) for repositioning the curvature plane by repositioning of the medical object. The instruction in this case may be provided relative to the positioning of the curvature plane at that moment and/or relative to a positioning of the medical object at that moment. For example, the instruction may have a movement specification and/or control specification for translation and/or rotation of the medical object. This enables the positioning of the curvature plane to be adjusted, especially in accordance with the pre-operative planning data.

In accordance with a second variant, the workflow note may have an instruction for adjusting the curvature of the distal section (e.g., a control specification and/or a required curvature parameter). In one embodiment, the workflow note may include a control specification for manipulation of the adjustment element (e.g., of a cable and/or of a rotary grip). The curvature of the distal section is able to be adjusted by the manipulation of the adjustment element. As an alternative or in addition, the workflow note may have the required curvature parameter. The required curvature parameter may specify a curvature direction and/or a curvature angle and/or a curvature path and/or a curvature geometry of the distal section of the medical object (e.g., with regard to the curvature plane).

The form of embodiment may make possible an intuitive repositioning of the curvature plane.

In a further form of embodiment of the method, the workflow note may be provided as a function of the identified positioning of the curvature plane and the planning data.

In one embodiment, the workflow note may be provided as a function of the identified positioning (e.g., at that moment) of the curvature plane and of the planning information for positioning of the medical object (e.g., the path planning and/or the target object and/or the required positioning and/or the required curvature for the medical object). As an alternative or in addition, the workflow note may be provided as a function of the identified positioning of the curvature plane and the pre-operative planning image.

The form of embodiment may make possible an intuitive repositioning of the curvature plane according to the planning data (e.g., a specification for a planning positioning of the curvature plane).

In a further version of the method, the acts a) to c) may be carried out repeatedly until an abort condition occurs.

The abort condition may have a predetermined (e.g., maximum) number of repetitions of the acts a) to c). The abort condition occurs on reaching or exceeding the predetermined number of repetitions. As an alternative or in addition, the abort condition may have a predetermined (e.g., maximum) duration for the repeated carrying out of the acts a) to c). The abort condition occurs on reaching or exceeding the predetermined duration. As an alternative or in addition, the abort condition may occur when the distal section is arranged at the target object and/or on reaching the required positioning and/or the required curvature for the medical object.

The present embodiments, in a second aspect, relate to a medical object, having at least one distal section, that is able to be curved parallel to a curvature plane, and a curvature marker structure with a display element (e.g., one visible to imaging) that is configured to quantify the curvature of the distal section at that moment.

The advantages of the medical object essentially correspond to the advantages of the method for provision of a curvature parameter of the present embodiments. Features, advantages, or alternate forms of embodiment mentioned here may likewise also be transferred to the other subject matter and vice versa.

In a further form of embodiment of the medical object, the medical object may further have an adjustment element that is configured for adjusting the curvature of the distal section. The adjustment element may have an indicator structure. In this case, a relative positioning between the indicator structure and the curvature marker structure may change on adjustment of the curvature of the distal section. The relative positioning between the indicator structure and the curvature marker structure may quantify the curvature of the distal section at that moment.

In one embodiment, the medical object may have an adjustment element that is configured for adjusting the curvature of the distal section. The adjustment element may further have an indicator structure as its display element that is visible to imaging (e.g., opaque to X-rays). In this case, a relative positioning between the indicator structure and the curvature marker structure may change during an adjustment of the curvature of the distal section. The relative positioning between the indicator structure and the curvature marker structure may quantify the curvature of the distal section at that moment. In one embodiment, the indicator structure and the curvature marker structure may be arranged on the distal section of the medical object. Further, the curvature marker structure and the indicator structure may be configured visible to imaging (e.g., opaque to X-rays).

For example, the indicator structure may have a scale (e.g., labelling and/or line scale and/or point scale), and the curvature marker structure may have a pointer (e.g., an arrow and/or a line and/or a point), or vice versa. In this case, the positioning of the pointer with regard to the scale at that moment may quantify the curvature at that moment. For example, the scale or the pointer may be arranged on the adjustment element (e.g., attached to the adjustment element) or be integrated at least partly (e.g., completely) in the adjustment element.

The form of embodiment may make possible a quantitative identification of the curvature of the distal section at that moment with the aid of an image of the indicator structure and of the curvature marker structure.

In a third aspect, the present embodiments relate to a system including a medical imaging device and a provision unit. In this case, the imaging device is configured for capturing medical imaging data, having an image of a medical object, that is arranged at least partly in an examination object. At least one distal section of the medical object is further able to be curved parallel to a curvature plane. The provision unit is configured to identify a positioning of the curvature plane with the aid of the image data and to provide a curvature parameter, having an indication for positioning of the curvature plane.

The medical imaging device for recording the image data may include a medical X-ray device (e.g., a medical C-arm X-ray device and/or a cone-beam computed tomography system (cone-beam CT, CBCT)), and/or a computed tomography (CT) system and/or a magnetic resonance tomography (MRT) system and/or a positron emission tomography (PET) system and/or an ultrasound device.

The advantages of the system essentially correspond with the advantages of the method of the present embodiments for provision of a curvature parameter. Features, advantages, or alternate forms of embodiment mentioned here may likewise also be transferred to the other subject matter and vice versa.

In a fourth aspect, the present embodiments relate to a computer program product with a computer program that is able to be loaded directly into a memory of a provision unit, with program sections for carrying out all acts of a method of the present embodiments for provision of a curvature parameter when the program sections are executed by the provision unit. The computer program product in this case may include software with a source code that still has to be compiled and linked or that only has to be interpreted, or may include executable software code that only has to be loaded into the provision unit for execution. The computer program product enables the method for provision of a curvature parameter by the provision unit to be carried out rapidly, identically repeatably, and robustly. The computer program product is configured so that the computer program product may carry out the method acts of the present embodiments by the provision unit.

The computer program product is stored, for example, on a computer-readable memory medium or held on a network or server, from where the computer program product may be loaded into the processor of a provision unit, so that the provision unit may be configured directly connected or be configured as part of the provision unit. Further, control information of the computer program product may be stored on an electronically readable data medium (e.g., a non-transitory-computer-readable storage medium). The control information of the electronically readable data medium may be configured such that, when the data medium is used in a provision unit, the control information carries out a method of the present embodiments. Examples of electronically readable data media are a DVD, a magnetic tape, or a USB stick, on which electronically readable control information (e.g., software) is stored. When this control information is read from the data medium and stored in a provision unit, all forms of embodiment of the method described above may be carried out.

A largely software-based realization has the advantage that provision units already previously used may be upgraded in a simple way by a software update in order to work according to the present embodiments. Such a computer program product, as well as the computer program, may, where necessary, include additional elements, such as, for example, documentation and/or additional components, as well as hardware components, such as, for example, hardware keys (e.g., dongles, etc.) for use of the software.

### BRIEF DESCRIPTION OF THE DRAWINGS

Example embodiments are shown in the drawings and will be described in greater detail below. The same reference characters will be used in different figures for the same features. In the figures:

FIGS. 1 to 5 show schematic diagrams of various forms of embodiment of a method for provision of a curvature parameter;

FIGS. 6 and 7 show schematic diagrams of various forms of embodiment of a medical object,

FIG. 8 shows a schematic diagram of an example of a graphical representation of the curvature parameter and of the image data; and

FIG. 9 shows a schematic diagram of a form of embodiment of a system.

### DETAILED DESCRIPTION

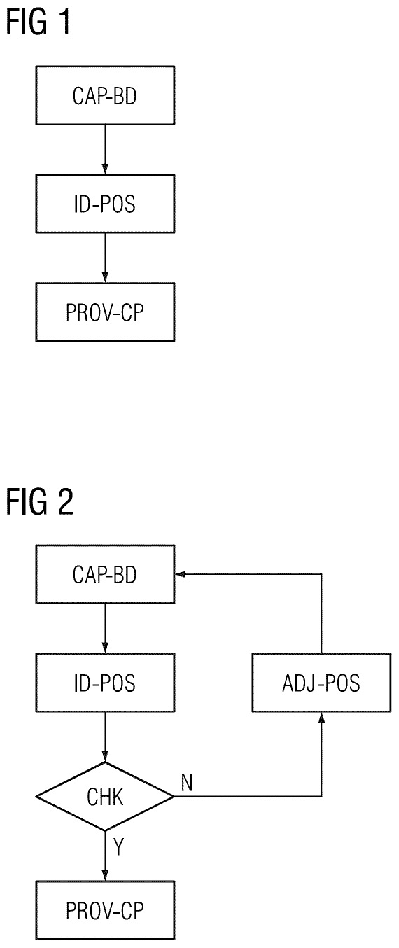

Shown schematically in FIG. 1 is a form of embodiment of a method for provision PROV-CP of a curvature parameter. In a first act a), medical image data, having an image of a medical object, that is arranged at least partly in an examination object is captured CAP-BD by a medical imaging device. In this case, at least one distal section of the medical object may be able to be curved parallel to a curvature plane. In a further act b), a positioning of the curvature plane may be identified ID-POS with the aid of the image data. In one embodiment, act b) may include an identification of a positioning of a curvature marker structure on the medical object that is depicted in the image data. Further, a curvature of the distal section of the medical object at that moment may be identified with the aid of the positioning of the curvature marker structure. In a further act c), the curvature parameter, having an indication for positioning of the curvature plane, may be provided PROV-CP. In this case, the curvature parameter having information about the curvature at that moment may be provided PROV-CP.

In one embodiment, the curvature marker structure may have a display element that is depicted in the image data and quantifies the curvature of the distal section at that moment. In this case, the provision PROV-CP of the curvature parameter may include a display of a graphical representation of the image data and of the curvature parameter by a visual display unit. In one embodiment, the graphical representation of the curvature parameter may include a virtual continuation of the distal section in accordance with its curvature at that moment.

In one embodiment, act c) may include a provision of a workflow note that has an instruction for repositioning of the medical object and/or an instruction for adjusting the curvature of the distal section. In one embodiment, the workflow note may be provided as a function of the identified positioning of the curvature plane and the planning data.

FIG. 2 shows a schematic diagram of a further form of embodiment of the method for provision PROV-CP of a curvature parameter. In this case, in act b), a check CHK may be made as to whether an imaging direction of the imaging device is aligned essentially at right angles to the curvature plane. In this case, if no N, a relative positioning between the medical object and the imaging device may be adjusted ADJ-POS, and the acts a) and b) carried out repeatedly. If yes Y, the method may be continued with act c).

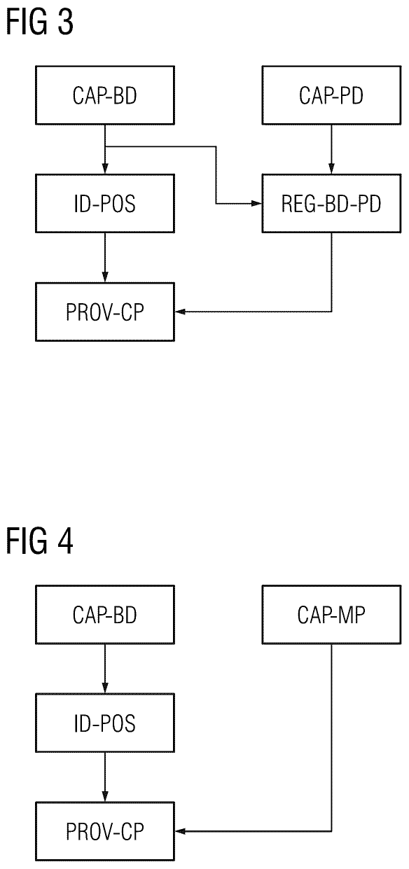

FIG. 3 shows a schematic diagram of a further form of embodiment of the method for provision PROV-CP of a curvature parameter. In this case, pre-operative planning data of the examination object may be received CAP-PD. The pre-operative planning data may be registered REG-BD-PD with the image data. The provision PROV-CP of the curvature parameter may further include a display of a graphical representation of the pre-operative planning data and of the curvature parameter. The planning data may include a pre-operative planning image of the examination object. In this case, the graphical representation of the planning data may include a graphical representation of the registered planning image. The provision PROV-CP of the curvature parameter may also include a display of a graphical representation of the image data.

FIG. 4 shows a schematic diagram of a further form of embodiment of the method for provision PROV-CP of a curvature parameter. In this case, a material parameter for the medical object may be captured CAP-MP. Further, the virtual continuation may be provided based on the material parameter and the curvature at that moment.

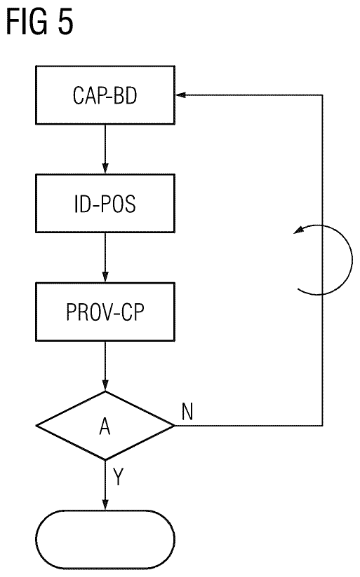

FIG. 5 shows a schematic diagram of a further form of embodiment of the method for provision PROV-CP of a curvature parameter. In this case, the acts a) to c) may be carried out repeatedly until the occurrence Y of an abort condition A.

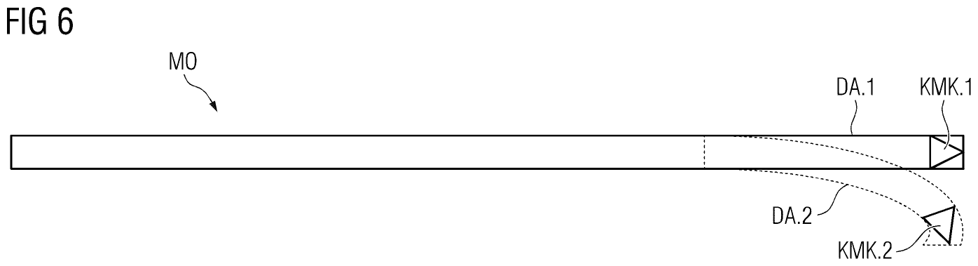

FIG. 6 shows a schematic diagram of a form of embodiment of a medical object MO. In this case, the medical object MO may have a distal section that is able to be curved parallel to a curvature plane (e.g., the plane of the drawing). Illustrated in FIG. 6 is the representation of an uncurved state DA.1 of the distal section and curved state DA.2 of the distal section. The medical object MO may further have a curvature marker structure KMK.1 and KMK.2 with a display element that is configured to quantify a curvature of the distal section at that moment. In this case, KMK.1 may show the distal section DA.1 in the uncurved state, and KMK.2 may show the distal section in the curved state DA.2.

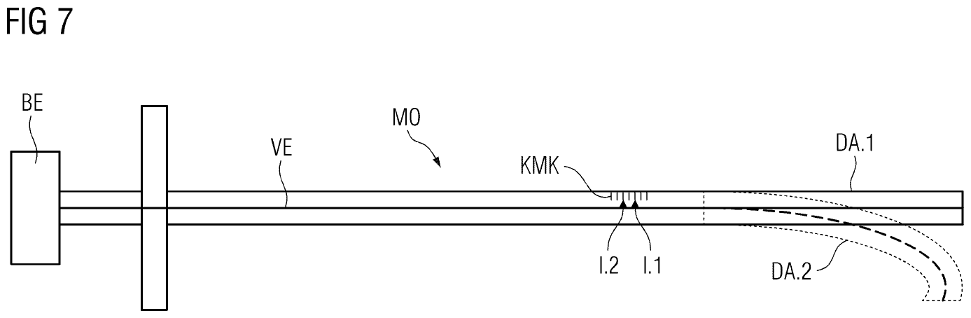

FIG. 7 shows a schematic diagram of a further form of embodiment of the medical object MO. Illustrated in FIG. 7 is the representation of an uncurved state DA.1 of the distal section and curved state DA.2 of the distal section. In this case, the medical object MO may have an adjustment element VE that is configured for adjusting the curvature of the distal section (e.g., a cable). The adjustment element VE may further have an indicator structure. In FIG. 7, the diagram illustrates a first positioning I.1 of the indicator structure in the uncurved state DA.1 of the distal section and a second positioning I.2 of the indicator structure in the curved state DA.2 of the distal section. During an adjustment of the curvature of the distal section (e.g., by manipulation of an operating element BE at a proximal section of the medical object MO), a relative positioning between the indicator structure and the curvature marker structure KMK may change. In this case, the relative positioning between the indicator structure and the curvature marker structure KMK may quantify curvature of the distal section at that moment. The curvature marker structure KMK may have a scale, for example.

As an alternative or in addition, a curvature of the medical object MO set at that moment may be captured by a sensor (e.g., an electromagnetic and/or optical and/or acoustic and/or mechanical sensor; not shown here) and may be able to be transmitted over a data interface (e.g., wirelessly) to a provision unit. In this case, the sensor may be arranged, for example, on the proximal section of the medical object MO (e.g., the operating element).

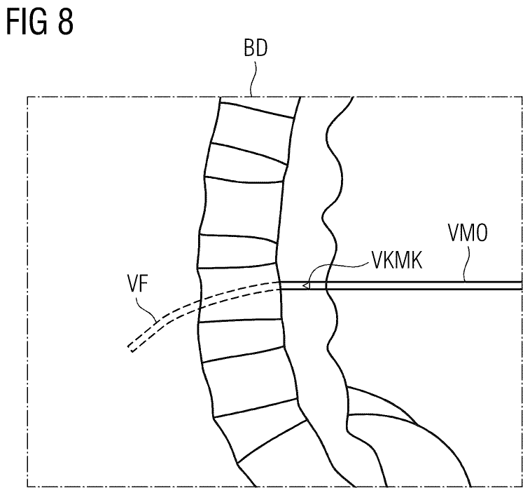

FIG. 8 shows a schematic diagram of an example of a graphical representation BD of the curvature parameter and the image data. In this case, the image data may have an image of the medical object VMO and the curvature marker structure VKMK. Further, the graphical representation BD may include a virtual continuation VF of the distal section of the medical object MO in accordance with its curvature at that moment.

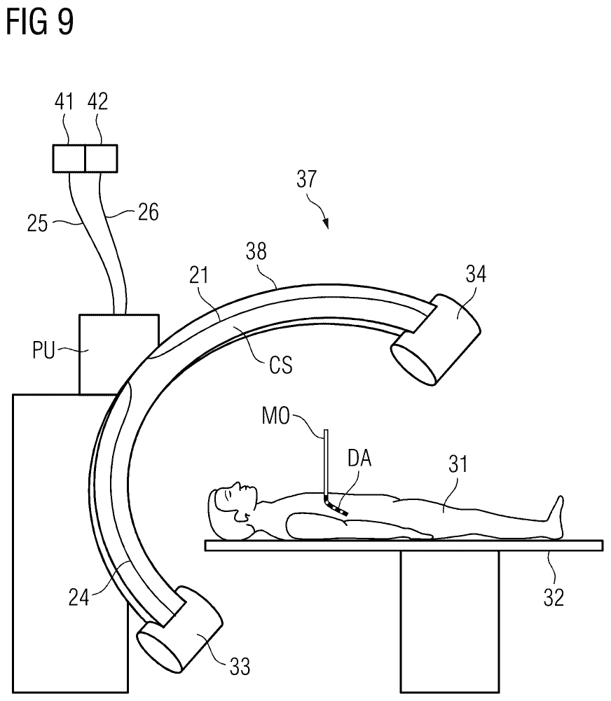

FIG. 9 shows a schematic diagram of one embodiment of a system. The system may include a medical imaging device (e.g., a medical C-arm X-ray device 37) and a provision unit PU. In this case, the imaging device may be configured for capturing the medical imaging data CAP-BD, having the image of the medical object MO, which is arranged at least partly in the examination object 31. At least the distal section of the medical object MO may further be able to be curved parallel to the curvature plane. The provision unit PU may be configured to identify ID-POS the positioning of the curvature plane with the aid of the image data and the curvature parameter, having the indication for providing PROV-CP for positioning of the curvature plane.

The C-arm X-ray device 37 can have an x-ray detector 34 and an X-ray source 33 that are arranged in a defined arrangement on a C arm 38. The C arm 38 of the C-arm X-ray device 37 may be supported about one or more axes. For example, the defined arrangement of X-ray source 33 and X-ray detector 34 may be supported movably about a center of rotation. For capturing CAP-BD the medical imaging data (e.g., at least one projection image) of the examination object 31 positioned on a patient support apparatus 32, and of the medical object MO arranged at least partly therein (e.g., of the distal section DA), the provision unit PU may send a signal 24 to the X-ray source 33. In response, the X-ray source 33 may emit an X-ray bundle. When the X-ray bundle, after interacting with the examination object 31, strikes a surface of the X-ray detector 34, the X-ray detector 34 may send a signal 21 to the provision unit PU. The processing unit PU may capture the image data CAP-BD with the aid of the signal 21.

In one embodiment, the system may have a visual display unit 41. The visual display unit 41 may include a screen and/or monitor and/or projector and/or video headset that are configured to provide the curvature parameter (e.g., to display a graphical representation of the curvature parameter and/or of the image data and/or of the registered planning data and/or of the workflow note). For this, the provision unit PU may provide a corresponding signal 25 to the visual display unit 41. The system may further have an input unit 42 (e.g., a keyboard). The input unit 42 may be integrated into the visual display unit 41 (e.g., in a capacitive and/or resistive input display). The input unit 42 can be configured for capturing a user input. For this, the input unit 42 may, for example, send a signal 26 to the provision unit PU. The capturing of the image data CAP-BD may be controlled with the aid of the user input.

The schematic diagrams contained in the described figures do not depict any scale or size relationship.

It is pointed out in conclusion that the method described in detail above as well as the apparatuses shown merely involve example embodiments that may be modified by the person skilled in the art in a very wide variety of ways, without departing from the field of the invention. Further, the use of the indefinite article “a” or “an” does not exclude the features concerned also being able to be present a number of times. Likewise, the terms “unit” and “element” do not exclude the components concerned consisting of a number of interacting part components that, where necessary, may also be spatially distributed.

The expression “based on” may be understood in the context of the present application, for example, in the sense of the expression “using”. For example, a formulation according to which a first feature is created (e.g., alternatively, established, determined, etc.) based on a second feature does not exclude that the first feature may be created (e.g., alternatively, established, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for providing a curvature parameter, the method comprising:
capturing medical imaging data by a medical imaging device, having an image of a medical object, the medical object being arranged at least partly in an examination object,
wherein at least one distal section of the medical object is curveable parallel to a curvature plane;
identifying a positioning of the curvature plane with the aid of the medical imaging data; and
providing the curvature parameter having an indication for positioning of the curvature plane.

2. The method of claim 1, wherein identifying the positioning of the curvature plane comprises checking whether an imaging direction of the imaging device is essentially aligned at right angles to the curvature plane,
wherein when, based on the checking, the imaging direction of the imaging device is not essentially aligned at right angles to the curvature plane, the method further comprises:
adjusting a relative positioning between the medical object and the imaging device; and
carrying out the capturing and the identifying of the positioning of the curvature plane repeatedly.

3. The method of claim 1, wherein identifying the positioning of the curvature plane comprises identifying a positioning of a curvature marker structure on the medical object that is depicted in the medical imaging data.

4. The method of claim 3, wherein a curvature of a distal section of the at least one distal section of the medical object at that moment is identified with the aid of the positioning of the curvature marker structure, and
wherein the curvature parameter is provided, having information about the curvature at that moment.

5. The method of claim 4, wherein the curvature marker structure has a display element that is depicted in the medical imaging data, and quantifies the curvature of the distal section at that moment, and
wherein providing the curvature parameter comprises displaying a graphical representation of the medical imaging data and of the curvature parameter by a visual display unit.

6. The method of claim 4, wherein the graphical representation of the curvature parameter comprises a virtual continuation of the distal section in accordance with the curvature at that moment.

7. The method of claim 6, further comprising capturing a material parameter for the medical object,
wherein the virtual continuation is provided based on the material parameter and the curvature at that moment.

8. The method of claim 1, further comprising:
receiving pre-operative planning data of the examination object; and
registering the pre-operative planning data with the medical imaging data,
wherein providing the curvature parameter comprises displaying a graphical representation of the registered pre-operative planning data and of the curvature parameter.

9. The method of claim 8, wherein the pre-operative planning data comprises a pre-operative planning image of the examination object,
wherein the graphical representation of the pre-operative planning data comprises a graphical representation of the registered pre-operative planning image,
wherein providing the curvature parameter further comprises displaying a graphical representation of the medical imaging data.

10. The method of claim 8, wherein providing the curvature parameter comprises providing a workflow note that has an instruction for repositioning the curvature plane by repositioning of the medical object, an instruction for adjusting the curvature of the distal section, or a combination thereof.

11. The method of claim 10, wherein the workflow note is provided as a function of the identified positioning of the curvature plane and the planning data.

12. The method of claim 1, wherein the capturing, the identifying, and the providing are carried out repeatedly until the occurrence of an abort condition.

13. A medical object comprising:
at least one distal section that is curveable parallel to a curvature plane; and
a curvature marker structure with a display element that is configured to quantify a curvature of a distal section of the at least one distal section at that moment.

14. The medical object of claim 13, further comprising:
an adjustment element that is configured for adjusting the curvature of the distal section,
wherein the adjustment element comprises an indicator structure,
wherein a relative positioning between the indicator structure and the curvature marker structure changes during an adjustment of the curvature of the distal section, and
wherein the relative positioning between the indicator structure and the curvature marker structure quantifies the curvature of the distal section at that moment.

15. A system comprising:
a medical imaging device; and
a provision unit,
wherein the medical imaging device is configured for capturing medical imaging data, having an image of a medical object that is at least partly arranged in an examination object,
wherein at least one distal section of the medical object is curveable parallel to a curvature plane,
wherein the provision unit is configured to:
identify a positioning of the curvature plane with the aid of the image data; and
providing a curvature parameter, having an indication for positioning of the curvature plane.
